(19) United States
(12) Patent Application Publication
(10) Pub. No.: US 2026/0069841 A1
(43) Pub. Date: Mar. 12, 2026

(54) ADJUSTABLE WEARABLE VAGINAL DILATION DEVICE

(71) Applicant: TONGJI HOSPITAL AFFILIATED TO TONGJI MEDICAL COLLEGE OF HUAZHONG UNIVERSITY OF SCIENCE & TECHNOLOGY, Hubei (CN)

(72) Inventors: Rong Liu, Hubei (CN); Haoming Tang, Hubei (CN)

(73) Assignee: TONGJI HOSPITAL AFFILIATED TO TONGJI MEDICAL COLLEGE OF HUAZHONG UNIVERSITY OF SCIENCE & TECHNOLOGY, Hubei (CN)

(21) Appl. No.: 19/322,536

(22) Filed: Sep. 8, 2025

(30) Foreign Application Priority Data
Sep. 9, 2024 (CN) 202411254536.6

(51) Int. Cl. A61M 29/00; A61M 39/22

(52) U.S. Cl. CPC A61M 29/00; A61M 39/22; A61M 2205/0216; A61M 2205/10; A61M 2205/3331; A61M 2205/502; A61M 2205/8206; A61M 2210/1475

(57) ABSTRACT

An adjustable wearable vaginal dilation device includes an outer-layer inflatable dilation assembly, an inner-layer inflation assembly, a flow guide mechanism, and an inflation control assembly. The outer-layer inflatable dilation assembly includes a plurality of independent airbags, the inner-layer inflation assembly penetrates through the interiors of the independent airbags, and a bottom baffle is sleeved at the bottom of the inner-layer inflation assembly; an elastic band is attached to a side edge of the bottom baffle, and an adhesive sheet is connected to a tail end of the elastic band; and the flow guide mechanism is mounted on the top of the outer-layer inflatable dilation assembly. According to the dilation device, dilation of different degrees can be provided for different areas of the vagina. Meanwhile, in the process of using the dilation device during the menstrual period, contamination caused by menstrual blood directly dripping on clothes can be avoided.

### CROSS-REFERENCE TO RELATED APPLICATION

This application claims the priority benefit of China application serial no. 202411254536.6, filed on Sep. 9, 2024. The entirety of the above-mentioned patent application is hereby incorporated by reference herein and made a part of this specification.

### TECHNICAL FIELD

This disclosure relates to the technical field of medical instruments, and in particular, to an adjustable wearable vaginal dilation device.

### BACKGROUND

Cenital vaginal atresia has a young age of onset, and it is most commonly seen that obstruongction of menstrual blood flows out after menarche (11-15 years old), leading to lower abdominal pain and subsequent medical consultation. The wearable vaginal dilation device can be used for vaginal dilation following vaginal reconstruction surgery for the congenital vaginal atresia. Through the use of a vaginal dilation apparatus for continuous dilation treatment, combined with medication-assisted therapy, the goal of providing gradual vaginal dilation treatment for patients with congenital vaginal atresia as they age can be achieved.

In the prior art, wearable therapeutic devices used for vaginal dilation can achieve the purpose of dilation simply by directly inserting a cylindrical instrument into the vagina. If a regulation function needs to be provided, it can be achieved directly through an inflatable elastic airbag: the inflation size of the airbag is controlled by inflating with gas, so that the dilation degree and state can be adjusted. However, the adjustment in the existing solution directly drives the entire dilation device to perform synchronous and uniform regulation, which results in the inability to perform targeted local dilation treatment for patients of different ages and body types. The regulation range is not precise enough. On the other hand, due to the need for auxiliary medication, the medicine liquid needs to be flushed during the subsequent wearable treatment process, which leads to frequent removal of the device, increasing the difficulty of treatment and exacerbating the patient's suffering during the treatment stage. Meanwhile, during the inflation process, the difficulty of controlling the inflation is also relatively high.

### SUMMARY

In view of the foregoing shortcomings in the prior art, this disclosure is intended to provide an adjustable wearable vaginal dilation device for solving the problems mentioned in the background. According to this disclosure, a gradual dilation treatment within a small range can be achieved, the discomfort of the treatment process is further reduced, contamination caused by directly dripping onto clothes is avoided, and internal flushing can be achieved, featuring higher safety and convenient operation.

To achieve the above objectives, this disclosure is implemented through the following technical solution: an adjustable wearable vaginal dilation device, including a dilation device body, where the dilation device body includes an outer-layer inflatable dilation assembly, an inner-layer inflation assembly, a flow guide mechanism, and an inflation control assembly; the outer-layer inflatable dilation assembly includes a plurality of independent airbags, the inner-layer inflation assembly penetrates through the interiors of the independent airbags, and a bottom baffle is sleeved at the bottom of the inner-layer inflation assembly; an elastic band is attached to a side edge of the bottom baffle, and an adhesive sheet is connected to a tail end of the elastic band; the flow guide mechanism is mounted on the top of the outer-layer inflatable dilation assembly, the interior of the flow guide mechanism is filled with liquid-absorbing cotton, a top-layer flushing hole is formed in the middle of the flow guide mechanism, and a central axis of the inner-layer inflation assembly coincides with a central axis of the top-layer flushing hole; and an inflation tube is inserted into a bottom end of the inner-layer inflation assembly, and a tail end of the inflation tube is connected to the inflation control assembly.

Further, the inner-layer inflation assembly includes a middle-layer sleeve and an inner-layer sleeve, and an inflation interlayer is formed between the middle-layer sleeve and the inner-layer sleeve; an inflation hole is formed in the top of a side edge of the middle-layer sleeve, and a flushing channel is arranged inside the inner-layer sleeve; and a docking hole is formed in the top of the flushing channel.

Further, the inflation holes are annularly and evenly distributed on a surface of the middle-layer sleeve, the inflation interlayer is in communication with the interior of the outer-layer inflatable dilation assembly through the inflation holes, and the bottom of the inner-layer sleeve is embedded with a plug-in sleeve shell.

Further, a bottom-layer flushing hole is formed in the bottom of the plug-in sleeve shell, a communication hole is formed in the bottom of the inflation interlayer, an air valve is mounted at the bottom of the communication hole, one end of the inflation tube is connected to the interior of the air valve, and an air tube connector is mounted on a surface of the inflation tube.

Further, the outer-layer inflatable dilation assembly includes the independent airbag and an outer-layer sleeve, an inner side of each independent airbag is attached to a surface of the outer-layer sleeve, an air inlet hole is formed in the outer-layer sleeve, and each independent airbag is in communication with the interior of the inflation interlayer after sequentially passing through the air inlet hole and the inflation hole.

Further, an inner wall of the outer-layer sleeve is attached to the surface of the middle-layer sleeve, each independent airbag has the same thickness, and the adjacent independent airbags are extruded and attached to each other.

Further, the flow guide mechanism includes an arc-shaped top and the liquid-absorbing cotton, a vertical rod is integrally formed at the bottom of the arc-shaped top, an extrusion plate is sleeved on a surface of the vertical rod, and a top end of the liquid-absorbing cotton is integrally bonded with the bottom of the arc-shaped top.

Further, the bottom of the liquid-absorbing cotton is integrally bonded with a surface of the extrusion plate, the top-layer flushing hole is formed in the middle of the arc-shaped top, and a liquid discharge cavity is formed between the topmost independent airbag and the arc-shaped top.

Further, the extrusion plate moves up and down inside the liquid discharge cavity, a bottom end of the vertical rod is bonded to a surface of the topmost independent airbag, and after the inner-layer inflation assembly moves upward, the extrusion plate is lifted upward.

Further, the inflation control assembly includes a control panel and an air pump, an electric proportional valve is connected to one end of the inflation tube, the electric proportional valve is connected to the air pump through a pipeline, an emergency stop switch and a microcontroller are mounted on a surface of the inflation control assembly, the microcontroller is electrically connected to the control panel through a cable, and a battery is further mounted inside the inflation control assembly.

Beneficial effects of this disclosure are as follows:
1. The adjustable wearable vaginal dilation device can provide different degrees of dilation for different vaginal areas. Meanwhile, the gradual dilation treatment within a small range can be achieved, and the discomfort of the treatment process is further reduced.
2. The adjustable wearable vaginal dilation device absorbs medicine liquid or menstrual blood through the liquid-absorbing cotton at the end, avoiding contamination caused by directly dripping onto clothes and improving the wearing experience. Meanwhile, the flushing liquid will not be blocked in the flushing stage, ensuring that the flushing liquid can be smoothly discharged from the body during the flushing process, which takes into account the effects of adsorption and flow guiding.
3. The adjustable wearable vaginal dilation device enables internal flushing, making the flushing more thorough. The flushing channel can also be used for draining internal medicinal liquid, facilitating medicine treatment. Frequent removal and wearing are not needed, so that the treatment experience is further optimized.
4. According to this disclosure, the speed of suction and deflation can be set before the operation of the machine through speed-up and speed-down buttons of the control panel. In case of an emergency, a user can cut off the entire circuit through the emergency stop switch to achieve emergency braking, featuring higher safety and convenient operation.

### BRIEF DESCRIPTION OF THE DRAWINGS

FIG. 1 is a schematic structural diagram of an external shape of an adjustable wearable vaginal dilation device according to this disclosure.

FIG. 2 is an internal cross-sectional view of this disclosure.

FIG. 3 is an enlarged view of a region A in FIG. 2.

FIG. 4 is a schematic structural diagram of an inner-layer inflation assembly part according to this disclosure.

FIG. 5 is an enlarged view of a region B in FIG. 2.

FIG. 6 is a schematic diagram of an intelligent control equipment part of a vaginal dilation device according to this disclosure.

### DETAILED DESCRIPTION OF THE EMBODIMENTS

To easily understand the technical means, creative features, objectives, and efficacy achieved by this disclosure, this disclosure will be further described below in combination with embodiments.

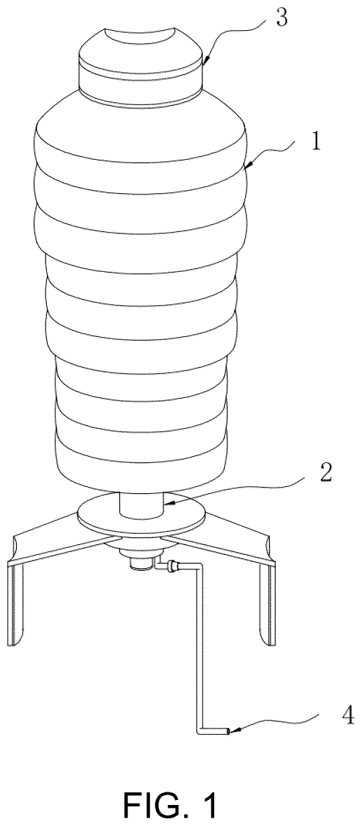

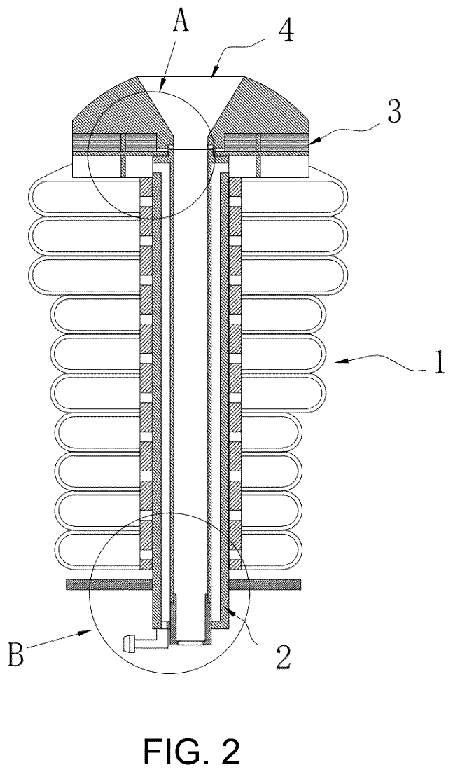

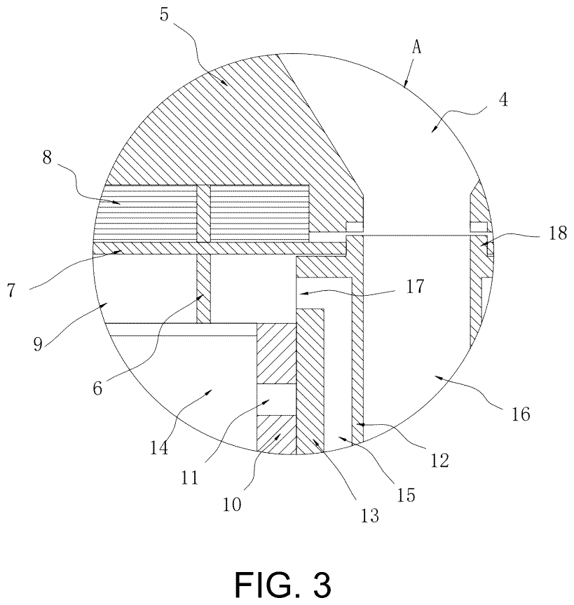

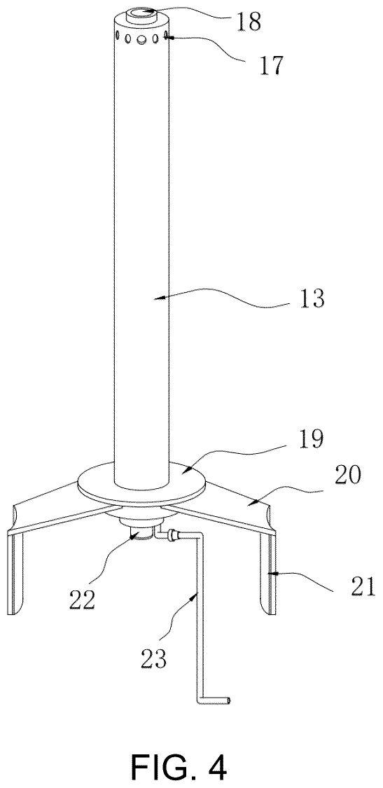

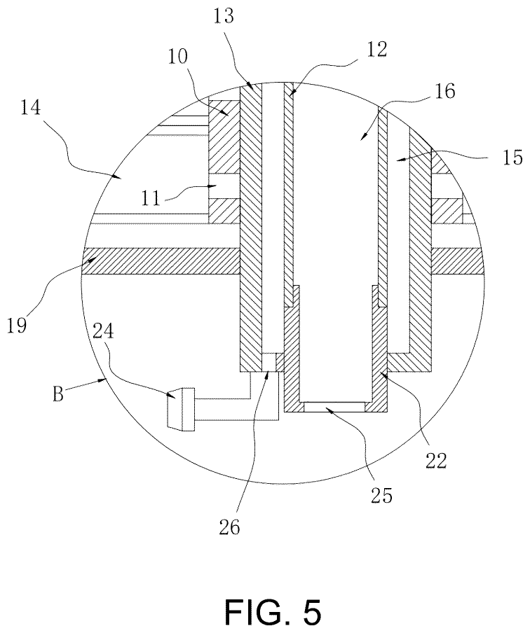

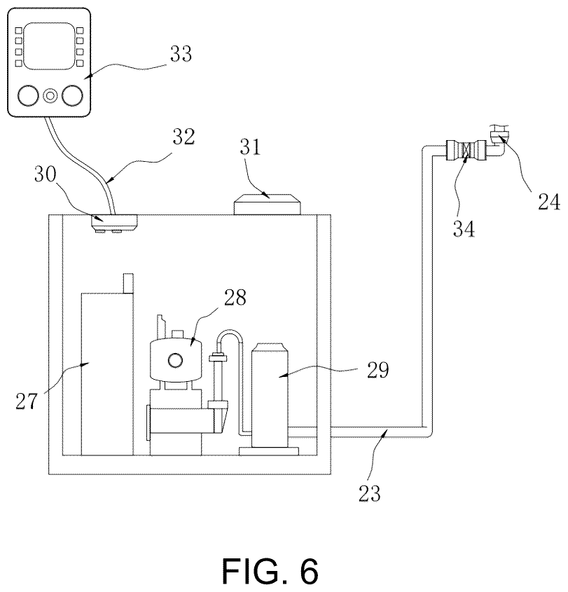

Referring to FIG. 1 to FIG. 6, this disclosure provides the following technical solution: an adjustable wearable vaginal dilation device, including a dilation device body, where the dilation device body includes an outer-layer inflatable dilation assembly 1, an inner-layer inflation assembly 2, a flow guide mechanism 3, and an inflation control assembly; the outer-layer inflatable dilation assembly 1 includes a plurality of independent airbags 14, the inner-layer inflation assembly 2 penetrates through the interiors of the independent airbags 14, and a bottom baffle 19 is sleeved at the bottom of the inner-layer inflation assembly 2; an elastic band 20 is attached to a side edge of the bottom baffle 19, and an adhesive sheet 21 is connected to a tail end of the elastic band 20; the flow guide mechanism 3 is mounted on the top of the outer-layer inflatable dilation assembly 1, the interior of the flow guide mechanism 3 is filled with liquid-absorbing cotton 8, a top-layer flushing hole 4 is formed in the middle of the flow guide mechanism 3, and a central axis of the inner-layer inflation assembly 2 coincides with a central axis of the top-layer flushing hole 4; and an inflation tube 23 is inserted into a bottom end of the inner-layer inflation assembly 2, and a tail end of the inflation tube 23 is connected to the inflation control assembly. The adjustable wearable vaginal dilation device is used for treating congenital vaginal atresia. During the treatment period, flushing and medication application can be performed at any time, and the vaginal dilation device can also be used during the menstrual period. In this embodiment, a diameter of the outer-layer inflatable dilation assembly 1 is less than 2 cm, and a diameter of the middle flushing channel 16 is less than 1 cm.

According to this disclosure, during use, first, only the outer-layer inflatable dilation assembly 1 is embedded into the vagina of the patient, and then the inner-layer inflation assembly 2 is inserted upward from the bottom of the outer-layer inflatable dilation assembly 1. During the insertion process, the inner-layer inflation assembly is sequentially in communication with each independent airbag 14 on the outer-layer inflatable dilation assembly 1. When in communication, the independent airbag 14 can be inflated through the external inflation control assembly to expand it, and finally, when the required width is reached, the inflation can be stopped. The inner-layer inflation assembly 2 continues to be controlled to move upward to inflate the subsequent independent airbag 14 until the independent airbags 14 at different positions are all inflated to a required diameter sequentially, and then the state is maintained. Then, the device is fixed by adhering the adhesive sheet 21 at the bottom to the bottom surface of the underwear or the inner area of the pants. When it is necessary to flush the interior of the vagina during wearing, the inner-layer inflation assembly 2 is first lifted upward, and then the external flushing solution is injected from the bottom-layer flushing hole 25 of the inner-layer inflation assembly 2, and is sprayed out from the top-layer flushing hole 4. After completing the flushing, all the flushed solution is discharged, and then the inner-layer inflation assembly 2 is pulled down to make it flush with the top of the outer-layer inflatable dilation assembly 1.

In this embodiment, the inner-layer inflation assembly 2 includes a middle-layer sleeve 13 and an inner-layer sleeve 12, and an inflation interlayer 15 is formed between the middle-layer sleeve 13 and the inner-layer sleeve 12; an inflation hole 17 is formed in the top of a side edge of the middle-layer sleeve 13, and a flushing channel 16 is arranged inside the inner-layer sleeve 12; and a docking hole 18 is formed in the top of the flushing channel 16. The inflation holes 17 are annularly and evenly distributed on a surface of the middle-layer sleeve 13, the inflation interlayer 15 is in communication with the interior of the outer-layer inflatable dilation assembly 1 through the inflation holes 17, and the bottom of the inner-layer sleeve 12 is embedded with a plug-in sleeve shell 22. A bottom-layer flushing hole 25 is formed in the bottom of the plug-in sleeve shell 22, a communication hole 26 is formed in the bottom of the inflation interlayer 15, an air valve 24 is mounted at the bottom of the communication hole 26, one end of the inflation tube 23 is connected to the interior of the air valve 24, and an air tube connector 34 is mounted on a surface of the inflation tube 23. The interior can be flushed, and the flushing is more thorough. The flushing channel 16 can also be used for draining internal medicinal liquid, facilitating medicine treatment. Frequent removal and wearing are not needed, so that the treatment experience is further optimized.

Specifically, a plurality of independent airbags 14 separated from each other are used, the middle of each independent airbag 14 is connected as a whole through the outer-layer sleeve 10, and then the middle-layer sleeve 13 and the inner-layer sleeve 12 move up and down along the interior of the outer-layer sleeve 10, so that the inflation holes 17 on the middle-layer sleeve 13 can be sequentially docked with the corresponding air inlet holes 11 on an inner side of each independent airbag 14, and in this position, from the external inflation tube 23 again. After the air flow is filled in through the air pump 28, only the independent airbag 14 can be inflated and dilated, and the specific inflated size of the independent airbags 14 at different depth positions can be accurately controlled through this process, so that dilation of different degrees can be provided for different vaginal areas. Meanwhile, the gradual dilation treatment within a small range can be achieved, and the discomfort of the treatment process is further reduced.

In this embodiment, the outer-layer inflatable dilation assembly 1 includes the independent airbag 14 and an outer-layer sleeve 10, an inner side of each independent airbag 14 is attached to a surface of the outer-layer sleeve 10, an air inlet hole 11 is formed in the outer-layer sleeve 10, and each independent airbag 14 is in communication with the interior of the inflation interlayer 15 after sequentially passing through the air inlet hole 11 and the inflation hole 17. An inner wall of the outer-layer sleeve 10 is attached to the surface of the middle-layer sleeve 13, each independent airbag 14 has the same thickness, and the adjacent independent airbags 14 are extruded and attached to each other. Dilation of different degrees can be provided for different vaginal areas. Meanwhile, the gradual dilation treatment within a small range can be achieved, and the discomfort of the treatment process is further reduced.

Subsequently, when the device needs to be removed, the inner-layer inflation assembly 2 is directly pulled down slowly, so that the inflation holes 17 can be sequentially docked with each air inlet hole 11. In this case, the inflation tube 23 is in a disconnected state. Thus, after the inflation holes 17 are docked with the air inlet holes 11 on the independent airbag 14, the air inside the independent airbag 14 can be quickly discharged, enabling the independent airbag 14 to contract and reset.

In this embodiment, the flow guide mechanism 3 includes an arc-shaped top 5 and the liquid-absorbing cotton 8, a vertical rod 6 is integrally formed at the bottom of the arc-shaped top 5, an extrusion plate 7 is sleeved on a surface of the vertical rod 6, and a top end of the liquid-absorbing cotton 8 is integrally bonded with the bottom of the arc-shaped top 5. The bottom of the liquid-absorbing cotton 8 is integrally bonded with a surface of the extrusion plate 7, the top-layer flushing hole 4 is formed in the middle of the arc-shaped top 5, and a liquid discharge cavity 9 is formed between the topmost independent airbag 14 and the arc-shaped top 5. The extrusion plate 7 moves up and down inside the liquid discharge cavity 9, a bottom end of the vertical rod 6 is bonded to a surface of the topmost independent airbag 14, and after the inner-layer inflation assembly 2 moves upward, the extrusion plate 7 is lifted upward. Medicine liquid or menstrual blood is absorbed through the liquid-absorbing cotton 8 at the end, avoiding contamination caused by directly dripping onto clothes and improving the wearing experience. Meanwhile, the flushing liquid will not be blocked in the flushing stage, ensuring that the flushing liquid can be smoothly discharged from the body during the flushing process, which takes into account the effects of adsorption and flow guiding.

Specifically, the liquid-absorbing cotton 8 is arranged at the top, the flushing channel 16 is arranged inside the inner-layer sleeve 12, and the docking hole 18 is arranged at the top of the flushing channel 16. Through this structure, flushing and cleaning can be performed from the bottom-layer flushing hole 25 during the wearing and treatment process. The medicinal liquid that has been introduced enters the arc-shaped top 5 from the docking hole 18 and is finally injected upward from the top-layer flushing hole 4. In this case, since the inner-layer inflation assembly 2 is moved upward, the extrusion plate 7 is lifted upward, so that the liquid-absorbing cotton 8 is compressed, thereby opening the bottom of the liquid discharge cavity 9. The topmost independent airbag 14 will block the bottom channel. Therefore, the flushing medicine liquid ultimately flows from the liquid discharge cavity 9 to the interior of the inflation hole 17, and then flows into the bottom communication hole 26 along the inflation interlayer 15, and finally is discharged towards the outside.

During other time periods, part of the medicine liquid inside the vagina is absorbed by the top-layer liquid-absorbing cotton 8, and the internal medicinal liquid is drained downward along the flushing channel 16 only after oversaturation, thereby preventing direct adhesion to the clothes. In addition, when the device is worn during the menstrual period, the top-layer liquid-absorbing cotton 8 can be removed and filled into the interior of the plug-in sleeve shell 22, so that the menstrual blood can flow along the middle flushing channel 16 into the liquid-absorbing cotton 8 already located at the bottom layer, thereby avoiding contamination caused by directly dripping onto the clothes. The liquid-absorbing cotton can be replaced at any time.

In this embodiment, the inflation control assembly includes a control panel 33 and an air pump 28, an electric proportional valve 29 is connected to one end of the inflation tube 23, the electric proportional valve 29 is connected to the air pump 28 through a pipeline, an emergency stop switch 31 and a microcontroller 30 are mounted on a surface of the inflation control assembly, the microcontroller 30 is electrically connected to the control panel 33 through a cable 32, and a battery 27 is further mounted inside the inflation control assembly. The speed of suction and deflation can be set before the operation of the machine through speed-up and speed-down buttons of the control panel 33. In case of an emergency, a user can cut off the entire circuit through the emergency stop switch 31 to achieve emergency braking, featuring higher safety and convenient operation.

Specifically, the dilation device is directly connected to the external intelligent inflation control assembly. During inflation, the pump operates, and the inner-layer inflation assembly is manually controlled to dock with the independent airbags 14 at different positions respectively, and during this process, gas is delivered to the different independent airbags 14 through the air tube and the electric proportional valve 29. During deflation, the inner-layer inflation assembly is manually pulled to move downward, the electric proportional valve 29 is equipped with a built-in pressure relief valve, and the gas in each independent airbag 14 is sequentially released through the air tube and the pressure relief valve of the electric proportional valve 29.

Connection and disassembly between the inflation control assembly and the dilation device: when connected, an air exchange valve 24 is initially in the closed state, the air tube of the dilation device is connected to the air tube connector 34, and the air exchange valve 24 is opened to achieve communication between the inflation control assembly and the dilation device; and when disassembling, the air exchange valve 24 is closed first, and then the air tube of the dilation device is removed from the air tube connector 34, so that the separation between the dilation device and the inflation control assembly can be achieved.

The above shows and describes the basic principles, main features, and advantages of this disclosure. It will be apparent to those skilled in the art that this disclosure is not limited to the details of the above exemplary embodiments, and that this disclosure can be embodied in other specific forms without departing from the spirit or essential characteristics of this disclosure.

In addition, it should be understood that although the description is described according to implementations, not each implementation only contains an independent technical solution, the description is narrated in this manner for clarity only, those skilled in the art should treat the description as a whole, and the technical solutions in each embodiment can be appropriately combined to form other implementations that those skilled in the art can understand.

What is claimed is:

1. An adjustable wearable vaginal dilation device, comprising a dilation device body, wherein the dilation device body comprises an outer-layer inflatable dilation assembly, an inner-layer inflation assembly, a flow guide mechanism, and an inflation control assembly; the outer-layer inflatable dilation assembly comprises a plurality of independent airbags, the inner-layer inflation assembly penetrates through interiors of the independent airbags, and a bottom baffle is sleeved at the bottom of the inner-layer inflation assembly; an elastic band is attached to a side edge of the bottom baffle, and an adhesive sheet is connected to a tail end of the elastic band; the flow guide mechanism is mounted on the top of the outer-layer inflatable dilation assembly, an interior of the flow guide mechanism is filled with liquid-absorbing cotton, a top-layer flushing hole is formed in the middle of the flow guide mechanism, and a central axis of the inner-layer inflation assembly coincides with a central axis of the top-layer flushing hole; an inflation tube is inserted into a bottom end of the inner-layer inflation assembly, and a tail end of the inflation tube is connected to the inflation control assembly; the inner-layer inflation assembly comprises a middle-layer sleeve and an inner-layer sleeve, and an inflation interlayer is formed between the middle-layer sleeve and the inner-layer sleeve; an inflation hole is formed in a top of a side edge of the middle-layer sleeve, and a flushing channel is arranged inside the inner-layer sleeve; a docking hole (18) is formed in a top of the flushing channel; the outer-layer inflatable dilation assembly comprises the independent airbags and an outer-layer sleeve, an inner side of each of the independent airbags is attached to a surface of the outer-layer sleeve, an air inlet hole is formed in the outer-layer sleeve, and each of the independent airbags is in communication with an interior of the inflation interlayer after sequentially passing through the air inlet hole and the inflation hole; the flow guide mechanism comprises an arc-shaped top and the liquid-absorbing cotton; a vertical rod is integrally formed at the bottom of the arc-shaped top, and an extrusion plate is sleeved on a surface of the vertical rod; a top end of the liquid-absorbing cotton is integrally bonded with the bottom of the arc-shaped top, and the bottom of the liquid-absorbing cotton is integrally bonded with a surface of the extrusion plate; the top-layer flushing hole is formed in a middle of the arc-shaped top; a liquid discharge cavity is formed between a topmost one of the independent airbags and the arc-shaped top, and the extrusion plate moves up and down inside the liquid discharge cavity; a bottom end of the vertical rod is bonded to a surface of the topmost one of the independent airbags; and after the inner-layer inflation assembly moves upward, the extrusion plate is lifted upward.

2. The adjustable wearable vaginal dilation device according to claim 1, wherein the inflation holes are annularly and evenly distributed on a surface of the middle-layer sleeve, the inflation interlayer is in communication with an interior of the outer-layer inflatable dilation assembly through the inflation holes, and the bottom of the inner-layer sleeve is embedded with a plug-in sleeve shell.

3. The adjustable wearable vaginal dilation device according to claim 2, wherein a bottom-layer flushing hole is formed in a bottom of the plug-in sleeve shell, a communication hole is formed in a bottom of the inflation interlayer, an air valve is mounted at a bottom of the communication hole, one end of the inflation tube is connected to an interior of the air valve, and an air tube connector (34) is mounted on a surface of the inflation tube.

4. The adjustable wearable vaginal dilation device according to claim 1, wherein an inner wall of the outer-layer sleeve is attached to a surface of the middle-layer sleeve, each of the independent airbags has a same thickness, and adjacent ones of the independent airbags are extruded and attached to each other.

5. The adjustable wearable vaginal dilation device according to claim 1, wherein the inflation control assembly comprises a control panel and an air pump, an electric proportional valve is connected to one end of the inflation tube, the electric proportional valve is connected to the air pump through a pipeline, an emergency stop switch and a microcontroller are mounted on a surface of the inflation control assembly, the microcontroller is electrically connected to the control panel through a cable, and a battery is further mounted inside the inflation control assembly.
